(19) United States
(12) Patent Application Publication
(10) Pub. No.: US 2004/0185025 A1
(43) Pub. Date: Sep. 23, 2004

(54) Cosmetic compositions containing at least one alkylamphohydroxyalkylsulphonate amphoteric surfactant and at least one hydroxy acid, and uses thereof

(76) Inventor: Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/739,101

(22) Filed: Dec. 19, 2003

Related U.S. Application Data
(60) Provisional application No. 60/439,464, filed on Jan. 13, 2003.

(30) Foreign Application Priority Data
Dec. 19, 2002 (FR) 02 16197

(51) Int. Cl. A61K 7/075; A61K 7/08

(52) U.S. Cl. 424/70.21

(57) ABSTRACT

This disclosure relates to novel cosmetic compositions, for example detergent cosmetic compositions which comprise, in a cosmetically acceptable medium, at least one amphoteric surfactant chosen from alkylamphohydroxyalkylsulphonates and salts thereof, and at least 1% by weight of at least one hydroxy acid and/or derivatives thereof. These compositions can be used for washing and/or treating keratin materials such as the hair or the skin.

This disclosure also relates to improved cosmetic properties, such as disentangling, smoothing, volume, lightness, softness, flexibility and manageability of keratin fibers.

This application claims benefit of U.S. Provisional Application No. 60/439,464, filed Jan. 13, 2003.

The present disclosure relates to novel cosmetic compositions, such as detergent compositions, which comprise, in a cosmetically acceptable medium, at least one amphoteric surfactant chosen from alkylamphohydroxyalkylsulphonates and salts thereof, and at least 1% by weight of at least one hydroxy acid.

It is known to use detergent compositions, such as shampoos and shower gels, based essentially on standard surfactants, for example anionic, non-ionic and/or amphoteric surfactants, for cleansing and/or washing the hair and/or the skin. These compositions are applied to wet hair or skin and the lather generated by massaging or rubbing with the hands can allow, after rinsing with water, the removal of the diverse soiling sometimes present on the hair or the skin.

While these base compositions may have a certain amount of washing power, their intrinsic cosmetic properties nevertheless may still be quite poor, for example, due to the fact that the relatively aggressive nature of such a cleansing treatment may, in the long run, result in more or less pronounced damage to keratin materials, associated for instance, with the gradual removal of the lipids or proteins contained in and/or at the surface of the keratin materials.

Thus, to improve the cosmetic properties of the above detergent compositions, for example, those detergents intended to be applied to “sensitized” hair, i.e., hair that has been damaged and/or embrittled, for instance, due to the chemical action of atmospheric agents and/or hair treatments such as permanent-waving, dyeing or bleaching, it is known to introduce into these compositions additional cosmetic agents known as conditioners, which are intended mainly to repair or limit the harmful or undesirable effects that may be induced by the various treatments or attacking factors to which hair fibres can be subjected. These conditioners may also improve the cosmetic behaviour of natural hair.

Alkylamphohydroxyalkylsulphonate amphoteric surfactants have been recommended in detergent cosmetic compositions. They have been described, for example, in Patent Application No. WO 99/36054.

Hair washing compositions using these surfactants alone, however, may notnot result in satisfactory cosmetic properties.

Moreover, it is known to use hydroxy acids in cosmetic compositions, for example in shampoos, with the aim of improving the mechanical properties, for example, the strength. However, these hair washing compositions may not provide satisfactory cosmetic properties.

Thus, the present inventor sought to find detergent cosmetic compositions with improved cosmetic properties, for example, regarding the disentangling, smoothing, flexibility, volume, lightness, softness, and manageability of the hair.

The present inventor has found, surprisingly, that the combination of at least one hydroxy acid in an amount greater than or equal to 1% by weight and of at least one amphoteric surfactant chosen from alkylamphohydroxyalkylsulphonates allows at least one of such improved properties to be achieved.

The compositions as disclosed herein impart to keratin naterials, for example the hair, at least one improved cosmetic property chosen from easy disentangling, lightness, smoothness, volume, softness, manageability, and flexibility, without any sensation of feeling laden.

One aspect of the present disclosure is thus a cosmetic composition comprising, in a cosmetically acceptable medium, at least one amphoteric surfactant chosen from alkylamphohydroxyalkylsulphonates and salts thereof, and at least 1% by weight of at least one hydroxy acid.

Another aspect of the present disclosure relates to the use of at least one amphoteric surfactant as defined above, in, or for the manufacture of, a cosmetic composition comprising at least 1% by weight of at least one hydroxy acid.

Yet another aspect of the present disclosure relates to a process for treating keratin materials, such as the hair, comprising applying a cosmetic composition as disclosed herein to the keratin materials.

An aspect of the present disclosure is also the use of a composition as disclosed herein to improve at least one property chosen from disentangling, smoothing, volume, lightness, softness, flexibility and manageability.

As disclosed herein, the term “keratin materials” means the hair, the eyelashes, the eyebrows, the skin, the nails, mucous membranes and/or the scalp, for example, the hair.

The various aspects of the disclosure will now be described in detail. All the meanings and definitions of the compounds as disclosed herein are valid for all aspects of the invention.

The at least one amphoteric surfactant chosen from alkylamphohydroxyalkylsulphonates and salts thereof may be chosen from those of formula (I):

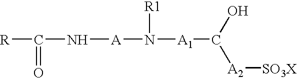

wherein:

R may be chosen from saturated and unsaturated, linear and branched hydrocarbon-based radicals comprising from 5 to 29 carbon atoms,

R, for example, may be chosen from mono- and polyunsaturated alkyl and alkenyl radicals comprising from 5 to 29 carbon atoms, such as from 7 to 22 carbon atoms and, for instance, from 9 to 17 carbon atoms,

R1 may be chosen from C₁-C₄ hydroxyalkyl radicals, such as hydroxyethyl,

A, A₁ and A₂, which may be identical or different, may be chosen from linear and branched C₁-C₁₀, such as C₁-C₃, divalent alkylene radicals,

X may be chosen from hydrogen and mineral and organic cations such as: that of an alkali metal (for example Na⁺ and K⁺), that of an alkaline-earth metal (Mg²⁺ and Ca²⁺), an NH₄⁺ ion, and ammonium ions derived from basic amino acids and/or from amino alcohols.

For example, as disclosed herein, the at least one amphoteric surfactant may be chosen from alkylamphohydroxyalkylsulphonates of formula (I) wherein R for instance, is chosen from saturated, linear and branched alkyl radicals comprising from 7 to 29 carbon atoms, such as from 7 to 22 carbon atoms.

When X is chosen from an ammonium ion derived from an alkanolamine, the alkanolamine may be chosen from monoethanolamine, diethanolamine, triethanolamine and 3-amino-1,2-propanediol. When X is chosen from an ammonium ion derived from an amine, the amine may be chosen from basic amino acids such as lysine, arginine, sarcosine, ornithine and citrulline.

For example, A may be the same as A₂ and both may be —CH₂CH₂—.

Further, for example, A₁ may be —CH₂—.

Still further, for example, X may be Na⁺.

Among the amphoteric surfactants of formula (I), non-limiting mention may be made of cocoyl amphohydroxypropyl sulphonate salts and for example, the sodium salt, such as the product sold under the name MIRANOL CSE by the company Rhodia Chimie, and palmitoyl amphohydroxypropyl sulphonate salts.

As disclosed herein, the at least one amphoteric surfactant chosen from alkylamphohydroxyalkylsulphonates and salts thereof, may be present in the composition in an amount ranging from 0.1% to 30% by weight, for example, from 1% to 20% by weight, and for further example, from 1.5% to 15% by weight, relative to the total weight of the composition.

As disclosed herein the cosmetic compositions also comprise at least 1% by weight of at least one hydroxy acid and/or derivatives thereof.

As disclosed herein, the term “acid derivatives” means the associated salts thereof, such as salts with an organic base or an alkali metal, and optionally the corresponding lactide thereof, i.e., the form obtained by self-esterification of the molecules.

The at least one hydroxy acid may comprise at least one mono- or polycarboxylic acid comprising at least one hydroxyl functional groups. The hydroxy acid may be chosen from α- and β-hydroxy acids. For example, the at least one hydroxy acid may be an α-hydroxy acid. The α and β positions reflect the fact that at least one of the hydroxyl functions occupies an α or β position relative to at least one of the carboxyl functions of the acid, i.e., is attached, respectively, either to the carbon bearing the hydroxyl function or to the carbon adjacent to the one bearing the carboxyl function. The acid may be present in the cosmetic composition in a form chosen from free acids, associated salts thereof (such as salts with organic bases and alkali metals), for example, depending on the final pH given to the composition, and optionally the corresponding lactides (i.e., the form obtained by self-esterification of the molecules).

Non-limiting examples of hydroxy acids as disclosed herein, include: salicylic acid, citric acid, lactic acid, methyllactic acid, phenyllactic acid, malic acid, mandelic acid, glycolic acid, tartronic acid, tartaric acid, gluconic acid, benzylic acid and 2-hydroxycaprylic acid. Other non-limiting examples of hydroxy acids that may be used as disclosed herein are mentioned in Patent Application No. EP-A-0 413 528, the teaching of which, with respect to hydroxy acids, is incorporated herein by reference.

For example, hydroxy acids that are cosmetically compatible with and acceptable for the hair, the skin and/or the scalp are used as disclosed herein.

For instance, according to one aspect of the present disclosure, the at least one hydroxy acid may be chosen from citric acid, tartaric acid and lactic acid.

According to another aspect of the cosmetic compositions as disclosed herein, the at least one hydroxy acid is present in the composition in an amount ranging from at least 1% by weight, such as at least 2% by weight, relative to the total weight of the composition. For example, the at least one hydroxy acid may be present in an amount ranging from 2% to 10% by weight, and such as from 3% to 6% by weight, relative to the total weight of the composition. These concentrations are markedly higher than those that may occasionally be found in known shampoos, wherein certain acids have been used solely for pH adjusting purposes.

For example, the weight ratio of the at least one amphoteric surfactant as disclosed herein to the at least one hydroxy acid as disclosed herein,may range from 0.1 to 20, such as from 0.5 to 15.

The compositions as disclosed herein may also comprise, for instance, at least one additional surfactant which may be present in the composition in an amount ranging from 0.1% to 40% by weight, such as from 3% to 30% and for further example, from 5% to 20%, by weight, relative to the total weight of the composition.

The at least one additional surfactant may be chosen from anionic, amphoteric and non-ionic surfactants.

Among the additional surfactants that may be used as disclosed herein, non-limiting mention may be made of the following:

(i) Anionic Surfactant(s):

In the context of the present disclosure, their nature is not an important feature.

Thus, as non-limiting examples of anionic surfactants that can be used, alone or as mixtures, as disclosed herein, mention may be made of salts, such as alkaline salts, for example sodium salts, ammonium salts, amine salts, amino alcohol salts and magnesium salts, of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates and N-acyltaurates, wherein the alkyl and/or acyl radicals of the above described compounds may comprise, for example, from 8 to 24 carbon atoms, and the aryl radicals may be chosen from, for instance, phenyl and benzyl groups. Also among the anionic surfactants that can be used, further non-limiting mention may be made of: fatty acid salts, such as the salts of oleic, ricinoleic, palmitic and stearic acids, coconut oil acid and hydrogenated coconut oil acid; acyl lactylates wherein the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, and polyoxyalkylenated (C₆-C₂₄) alkylamido ether carboxylic acids and their salts, for example, those comprising from 2 to 50 ethylene oxide groups.

For example, the at least one additional surfactant may be an anionic surfactant chosen from alkyl sulphate salts, cocoyl isethionate salts and alkyl ether sulphate salts.

For further example, the at least one additional surfactant may be an anionic surfactant chosen from sodium, magnesium and ammonium (C₁₂-C₁₄)alkyl sulphates; sodium, magnesium and ammonium (C₁₂-C₁₄)alkyl ether sulphates oxyethylenated with 2.2 mol of ethylene oxide; and sodium cocoyl isethionate.

(ii) Nonionic Surfactant(s):

The non-ionic surfactants are, themselves also, compounds that are known per se (see, for example, “Handbook of Surfactants” by M. R. Porter, published by Blackie & Son (Glasgow and London), 1991, pp. 116-178) and, in the context of the present disclosure, their nature is not an important feature. Thus, non-limiting examples of non-ionic surfactants that may be used as disclosed herein include: polyethoxylated, polypropoxylated and polyglycerolated fatty acids, alkylphenols, α-diols and alcohols having a fatty chain comprising, for example, 8 to 18 carbon atoms, it being possible for the number of ethylene oxide and/or propylene oxide groups to range, for example, from 2 to 50; for example, the number of glycerol groups may range from 2 to 30. Non-limiting mention may also be made of copolymers of ethylene oxide and of propylene oxide; condensates of ethylene oxide and of propylene oxide with fatty alcohols; polyethoxylated fatty amides having for example, from 2 to 30 mol of ethylene oxide; polyglycerolated fatty amides comprising, on average, from 1 to 5, such as from 1.5 to 4, glycerol groups; oxyethylenated fatty acid esters of sorbitan having from 2 to 30 mol of ethylene oxide; fatty acid esters of sucrose; fatty acid esters of polyethylene glycol; alkylpolyglycosides; N-alkyl-glucamine derivatives; amine oxides such as (C₁₀-C₁₄)alkylamine oxides; and N-acylaminopropylmorpholine oxides. For example, the at least one additional surfactant may be a non-ionic surfactant chosen from alkylpolyglycosides.

(iii) Amphoteric Surfactant(s):

Non-limiting examples of the at least one additional amphoteric surfactant, whose nature is not important in the context of the present disclosure, include those chosen from: aliphatic secondary and tertiary amine derivatives, wherein the aliphatic radical may be chosen from linear and branched chains comprising 8 to 22 carbon atoms and comprising at least one water-soluble anionic group, such as, for example, carboxylates, sulphonates, sulphates, phosphates and phosphonates); non-limiting mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

For example, among the amine derivatives, non-limiting mention may be made of the products sold under the name MIRANOL, as described in U.S. Pat. Nos. 2,528,378 and 2,781,354 and having the structures:

R₂—CONHCH₂CH₂—N(R₃)(R₄)(CH₂COO—) (2)

wherein: R₂ may be chosen from alkyl radicals derived from an acid R₂—COOH present in hydrolysed coconut oil, and from heptyl, nonyl and undecyl radicals, R₃ may be chosen from β-hydroxyethyl groups and R₄ is a carboxymethyl group; and

R₅—CONHCH₂CH₂—N(B)(D) (3)

wherein:

B is chosen from —CH₂CH₂OX′ groups,

D is chosen from —(CH₂)_{z}—Y′ groups, wherein z is an integer equal to 1 or 2,

X′ is chosen from —CH₂CH₂—COOH groups and hydrogen,

Y′ is chosen from —COOH and —CH₂—CHOH—SO₃H radicals,

R₅ is chosen from alkyl radicals derived from the carboxylic acids present in coconut oil and/or in hydrolyzed linseed oil, alkyl radicals, such as C₇, C₉, C₁₁, and C₁₃ alkyl radicals, a C₁₇ alkyl radical and its iso form, and an unsaturated C₁₇ radical.

These compounds are classified in the CTFA dictionary, 7^{th} edition, 1997, under the names disodium cocoamphodiacetate, disodium lauroamphodiacetate, disodium capryloamphodiacetate, disodium caproamphodiacetate, disodium cocoamphodipropionate, disodium lauroamphodipropionate, disodium caprylampho-dipropionate, disodium capryloamphodipropionate, lauroamphodipropionic acid, and cocoamphodipropionic acid.

By way of non-limiting example, mention may be made of the disodium cocoamphodiacetate sold under the trade name Miranol® C2M concentrate by the company Rhodia Chimie.

In the compositions as disclosed herein, mixtures of surfactants can be used, for instance, mixtures of anionic surfactants and mixtures of anionic surfactants and of amphoteric and/or nonionic surfactants.

As disclosed herein, the at least one anionic surfactant may be present in the composition in an amount ranging from 1% to 30% by weight, such as from 3% to 15% by weight, relative to the total weight of the composition.

As disclosed herein, the at least one additional amphoteric surfactant (other than the sulphonate compounds) and/or the at least one nonionic surfactant may be present in the composition in an amount ranging from 0.5% to 15% by weight, for example, ranging from 1% to 5% by weight, relative to the total weight of the composition.

The quantity and quality of the surfactants are those that are sufficient to give the final composition a satisfactory foaming power and/or detergent power.

In the composition as disclosed herein, the total amount of detergent surfactants may be present in the composition in an amount ranging from 4% to 50% by weight, such as ranging from 6% to 35% by weight, and for instance, ranging from 8% to 25% by weight, relative to the total weight of the composition.

According toone aspect of the present disclosure, the compositions as disclosed herein may further comprise at least one cationic polymer.

The cationic polymers that may be used as disclosed herein may be chosen from any of those already known per se as improving the cosmetic properties of hair treated with detergent compositions, for example, the cationic polymers described in Patent Application EP-A-0 337 354 and French Patent Application Nos. FR-A-2 270 846, 2 383 660, 2 598 611, 2 470 596 and 2 519 863.

For the purposes of the present disclosure, the term “cationic polymer” means any polymer comprising cationic groups and/or groups that may be ionizable into cationic groups.

Among the cationic polymers that may be used herein, non-limiting examples include: quaternary cellulose ether derivatives such as the products sold under the name “JR 400” by the company Amerchol; cyclopolymers, such as the homopolymers of diallyidimethylammonium salt and the copolymers of diallyldimethylammonium salt and of acrylamide, such as the chlorides, sold under the names “MERQUAT 100”, “MERQUAT 550” AND “MERQUAT S” by the company Ondeo; non-cellulose-based cationic polysaccharides, for instance guar gums modified with 2,3-epoxypropyltrimethyl-ammonium chloride, for example sold under the name ”JAGUAR C13S” by the company Rhodia Chimie.

It is also possible to use cationic polymers comprising repeating units of the formula:

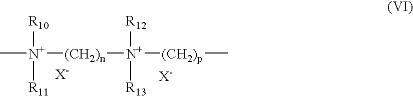

wherein, R₁₀, R₁₁, R₁₂ and R₁₃, which may be identical or different, are chosen from alkyl and hydroxyalkyl radicals comprising from 1 to 4 carbon atoms, n and p are integers ranging from 2 to 20, and X⁻ is an anion derived from mineral and/or organic acids.

As disclosed herein, the at least one cationic polymer may be present in the composition in an amount ranging from 0.001% to 10% by weight, such as from 0.005% to 5% by weight, and for instance, from 0.01% to 3% by weight, relative to the total weight of the final composition.

Additionally, the compositions as disclosed herein may further comprise at least one viscosity regulator, such as electrolytes and thickeners. Non-limiting mention may be made, for example, of sodium chloride, scleroglucans, xanthan gums, fatty acid alkanolamides, alkyl ether carboxylic acid alkanolamides optionally oxyethylenated with up to 5 mol of ethylene oxide, such as the product sold under the name “AMINOL A15” by the company Chem Y, crosslinked polyacrylic acids and acrylic acid/C₁₀-C₃₀ alkyl acrylate crosslinked copolymers. As disclosed herein, the at least one viscosity regulator may be present in the composition in an amount less than or equal to 10% by weight, relative to the total weight of the composition.

The compositions as disclosed herein may also comprise up to 5% by weight, relative to the total weight of the composition of nacreous agents and/or opacifiers that are known in the art. For instance, non-limiting examples include: coated and uncoated titanium oxides, sodium palmitate, magnesium palmitate, sodium stearate, sodium hydroxystearate, magnesium stearate, magnesium hydroxystearate, fatty-chain acyl derivatives such as ethylene glycol and polyethylene glycol monostearates and distearates, fatty-chain ethers (for example of C8-C30), for instance distearyl ether and 1-(hexa-decyloxy)-2-octadcanol, cyclodextrins, and fatty alcohols such as cetyl alcohol, stearyl alcohol and behenyl alcohol.

The compositions as disclosed herein may also comprise at least one foam synergist, such as C₁₀-C₁₈ 1,2-alkanediols and fatty alkanolamides derived from monoethanolamine and/or from diethanolamine.

The composition as disclosed herein may further comprise at least one additive chosen from fragrances, preserving agents, sunscreens, cationic surfactants, anionic, non-ionic and amphoteric polymers, proteins, protein hydrolyzates, ceramides, pseudoceramides, linear and branched C₁₆-C₄₀ fatty acids, such as 18-methyleicosanoic acid, vitamins, provitamins such as panthenol, plant oils, mineral oils and synthetic oils, antidandruff agents and any other additive conventionally used in cosmetics that does not affect the stability and the properties of the compositions as disclosed herein.

The at least one additive may be present in the composition as disclosed herein in an amount that may range from 0.001% to 50% by weight, relative to the total weight of the composition. The precise amount of each additive can be determined by a person of ordinary skill in the art, depending on the nature and function of each additive.

The cosmetically acceptable medium may comprise water, or a mixture of water and a cosmetically acceptable solvent such as a C₁-C₄ lower alcohol, for instance ethanol, isopropanol, tert-butanol and n-butanol; and alkylene glycols, for instance propylene glycol, and glycol ethers.

For example, the composition may comprise from 50% to 95% by weight of water relative to the total weight of the composition.

The detergent compositions as disclosed herein have a final pH ranging from 3 to 10. For example, the pH can range from 4 to 8. The pH may be adjusted to the desired value conventionally, by adding a base, such as organic or mineral bases, to the composition, for example aqueous ammonia, or primary, secondary or tertiary (poly)amines, for instance monoethanolamine, diethanolamine, triethanolamine, isopropanolamine and 1,3-propanediamine, or alternatively by adding an acid, such as carboxylic acids, for instance citric acid.

The compositions as disclosed herein may be used for washing and/or treating keratin materials such as the hair, the skin, the eyelashes, the eyebrows, the nails, the lips and the scalp; for example, the hair.

For example, the detergent compositions as disclosed herein may be chosen from shampoos, shower gels and bubble baths.

The compositions as disclosed herein may also be in the form chosen from rinse-out and leave-in conditioners; permanent-waving, hair-relaxing, dyeing and bleaching compositions; rinse-out compositions to be applied before and/or after dyeing, bleaching, permanent-waving and/or relaxing the hair; and compositions to be applied between the two steps of permanent-waving or hair-relaxing operation.

The compositions as disclosed herein may also be in the form of makeup-removing products.

The compositions as disclosed herein may also be in the form of gels, milks, creams, emulsions, thickened lotions and mousses, and the compositions may be used for the skin, the scalp, the nails, the eyelashes, the lips and, for example, the hair.

In one aspect of the present disclosure, the detergent compositions are foaming compositions and the foaming power of the compositions as disclosed herein, characterized by a foam height, is generally greater than 75 mm, for example, greater than 100 mm, measured according to the modified Ross-Miles method (NF T 73-404/ISO696). The modifications to the method are the following:

The measurement is performed at a temperature of 22° C. with osmosed water. The concentration of the solution is 2 g/L. The drop height is 1 m. The amount of composition dropped is 200 mL. The 200 mL of composition fall into a measuring cylinder with a diameter of 50 mm, containing 50 ml of the test composition. The measurement is performed 5 minutes after stopping the flow of the composition.

Another aspect of the present disclosure is a process for treating keratin materials such as the skin and the hair, comprising applying to the keratin materials a cosmetic composition as disclosed herein, and then optionally rinsing it out, such as with water. Thus, this process according to the present disclosure allows the treatment, care, washing and/or the removal of makeup from the skin, the hair and any other keratin material.

Throughout the text hereinabove and hereinbelow, the percentages expressed are on a weight basis.

The disclosure will now be illustrated more fully with the aid of the examples that follow, which cannot be considered as limiting it to the embodiments described. In the examples, AM means active material.

### EXAMPLE 1

Two shampoo compositions were prepared, one in accordance with the disclosure (composition A) and the other comparative (composition B):

| Composition | Disclosure | | Comparative | |
|---|---|---|---|---|
| Sodium lauryl ether sulphate (70/30 | 11.2 | g AM | 11.2 | g AM |
| C12/C14) containing 2.2 mol of ethylene | | | | |
| oxide | | | | |
| Sodium cocoylamphohydroxy- | 3.0 | g AM | — | |
| propylsulphonate, as an aqueous solution | | | | |
| containing 32% active material, sold under | | | | |
| the name Miranol CSE by the company | | | | |
| Rhodia | | | | |
| Sodium cocoamidoethyl(N-hydroxyethyl-N- | — | | 3.0 | g AM |
| carboxymethyl)glycinate, sold under the | | | | |
| name Miranol C2M by the company Rhodia | | | | |
| Hydroxyethylcellulose crosslinked with | 0.8 | g | 0.8 | g |
| epichlorohydrin and quaternized with | | | | |
| trimethlamine, sold under the name JR 400 | | | | |
| by the company Amerchol | | | | |
| Ethylene glycol distearate | 2.0 | g | 2.0 | g |
| Citric acid | 3.0 | g | 3.0 | g |
| Crosslinked polyacrylic acid | 0.2 | g | 0.2 | g |
| Preserving agents | qs | | qs | |
| Fragrance | qs | | qs | |
| Ammonia qs | pH 5.3 | | pH 5.3 | |
| Demineralized water qs | 100.0 | g | 100.0 | g |

A shampoo wash was performed by applying about 6 g of composition A to half a head of premoistened natural hair. The shampoo was worked into a lather and then rinsed out thoroughly with water. The hair was dried with a hairdryer.

The same procedure as above was performed with the comparative composition B on the other half of the head.

Experts compared the two half-heads in pairs.

A panel of experts evaluated the appearance of the dried hair and noted

greater ease of disentangling

greater smoothing (visual and to the touch).

The hair treated with composition A was significantly smoother and disentangled more easily than the hair treated with composition B.

### EXAMPLES 2 TO 4

The shampoo compositions below were prepared:

| Composition | Example 2 | | Example 3 | | Example 4 | |
|---|---|---|---|---|---|---|
| Sodium lauryl ether sulphate (70/30 C12/C14) containing 2.2 mol of ethylene oxide | 11.2 | g AM | 11.2 | g AM | 11.2 | g AM |
| Sodium cocoylamphohydroxypropylsulphonate, as an aqueous solution containing 32% | 3 | g AM | 3 | g AM | 3 | g AM |
| active material (MIRANOL CSE by the company Rhodia) | | | | | | |
| Hydroxyethylcellulose crosslinked with epichlorohydrin and quaternized with | 0.8 | g | 0.8 | g | 0.3 | g |
| trimethylamine (JR 400 by the company Amerchol) | | | | | | |
| Ethylene glycol distearate | 2 | g | 2 | g | 2 | g |
| Citric acid | 3 | g | 3 | g | 3 | g |
| Crosslinked polyacrylic acid | 0.2 | g | 0.2 | g | 0.2 | g |
| Preserving agents | qs | | qs | | qs | |
| Fragrance | qs | | qs | | qs | |
| Sodium hydroxide (NaOH) qs | — | | — | | pH 5.3 | |
| Aminomethylpropanol qs | — | | pH 5.3 | | — | |
| Aqueous ammonia qs | pH 5.3 | | — | | — | |
| Demineralized water qs | 100 | g | 100 | g | 100 | g |

The hair washed with these compositions was smooth, supple and manageable.

What is claimed is:

1. A cosmetic composition, comprising, in a cosmetically acceptable medium, at least one amphoteric surfactant chosen from alkylamphohydroxyalkylsulphonates and salts thereof, and at least 1% by weight, relative to the total weight of the composition, of at least one hydroxy acid and/or at least one derivative thereof.

2. The composition according to claim 1, wherein the at least one amphoteric surfactant is chosen from alkylamphohydroxyalkylsulphonates of formula (I): 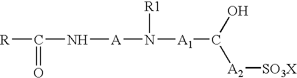
wherein:
R is chosen from saturated and unsaturated, linear and branched hydrocarbon-based radicals comprising from 5 to 29 carbon atoms,
R1 is chosen from C1-C4 hydroxyalkyl radicals,
A, A₁ and A₂, which may be identical or different, are chosen from linear and branched C₁-C₁₀ divalent alkylene radicals,
X is chosen from hydrogen and from mineral and organic cations.

3. The composition according to claim 2, wherein R is chosen from mono- and polyunsaturated alkyl and alkenyl radicals comprising from 5 to 29 carbon atoms.

4. The composition according to claim 3, wherein R is chosen from mono- and polyunsaturated alkyl and alkenyl radicals comprising from 7 to 22 carbon atoms.

5. The composition according to claim 4, wherein R is chosen from mono- and polyunsaturated alkyl and alkenyl radicals comprising from 9 to 17 carbon atoms.

6. The composition according to claim 1, wherein R1 is hydroxyethyl.

7. The composition according to claim 1, wherein A, A₁ and A₂, which may be identical or different, are chosen from linear and branched C₁-C₃ divalent alkylene radicals.

8. The composition according to claim 7, wherein A and A₂ are both equal to —CH₂CH₂—.

9. The composition according to claim 7, wherein A1 is —CH₂—.

10. The composition according claim 1 wherein the mineral and organic cations are chosen from alkali metals, alkaline-earth metals, an NH₄⁺ ion, and ammonium ions derived from basic amino acids and amino alcohols.

11. The composition according to claim 10, wherein the alkali metals are chosen from Na⁺ and K⁺.

12. The composition according to claim 10, wherein the alkaline-earth metals are chosen from Mg²⁺ and Ca²⁺.

13. The composition according to claim 1, wherein the at least one amphoteric surfactant is chosen from cocoyl amphohydroxypropylsulphonate salts and palmitoyl amphohydroxypropylsulphonate salts.

14. The composition according to claim 1, wherein the at least one amphoteric surfactant is present in the composition in an amount ranging from 0.1% to 30% by weight.

15. The composition according to claim 14, wherein the at least one amphoteric surfactant is present in the composition in an amount ranging from 1% to 20% by weight, relative to the total weight of the composition.

16. The composition according to claim 15, wherein the at least one amphoteric surfactant is present in the composition in an amount ranging from 1.5% to 15% by weight, relative to the total weight of the composition.

17. The composition according to claim 1, wherein the at least one hydroxy acid and/or at least one derivative thereof, is present in the composition in a form chosen from free acids, associated salts thereof, and corresponding lactides.

18. The composition according to claim 1, wherein the at least one hydroxy acid is chosen from α-hydroxy acids.

19. The composition according to claim 1, wherein the at least one hydroxy acid is chosen from β-hydroxy acids.

20. The composition according to claim 18, wherein the α-hydroxy acids are chosen from citric acid, lactic acid, methyllactic acid, phenyllactic acid, malic acid, mandelic acid, glycolic acid, tartronic acid, tartaric acid, gluconic acid, benzylic acid, and 2-hydroxycaprylic acid.

21. The composition according to claim 19, wherein the at least one β-hydroxy acid is salicylic acid.

22. The composition according to claim 1, wherein the at least one hydroxy acid is present in the composition in an amount ranging from 2% to 10% by weight, relative to the total weight of the composition.

23. The composition according to claim 22, wherein the at least one hydroxy acid is present in the composition in an amount ranging from 3% to 6% by weight, relative to the total weight of the composition.

24. The composition according to claim 1, further comprising at least one additional surfactant chosen from anionic, cationic, nonionic and amphoteric surfactants.

25. The composition according to claim 24, wherein the at least one additional surfactant is present in the composition in an amount ranging from 0.1% to 40% by weight, relative to the total weight of the composition.

26. The composition according to claim 25, wherein the at least one additional surfactant is present in the composition in an amount ranging from 3% to 30% by weight, relative to the total weight of the composition.

27. The composition according to claim 26, wherein the at least one additional surfactant is present in the composition in an amount ranging from 5% to 20% by weight, relative to the total weight of the composition.

28. The composition according to claim 1, further comprising at least one silicone.

29. The composition according to claim 1, further comprising at least one cationic polymer.

30. The composition according to claim 29, wherein the at least one cationic polymer is chosen from:
homopolymers of diallyldimethylammonium salt and copolymers of diallyldimethylammonium salt and of acrylamide,
quaternary cellulose ether derivatives,
non-cellulose-based cationic polysaccharides,
polymers comprising repeating units corresponding to the formula: 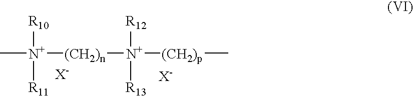
wherein, R₁₀, R₁₁, R₁₂ and R₁₃, which may be identical or different, are chosen from alkyl and hydroxyalkyl radicals comprising from 1 to 4 carbon atoms, n and p are integers ranging from 2 to 20, and X⁻ is an anion derived from mineral or organic acids.

31. The composition according to claim 29, wherein the at least one cationic polymer is present in the composition in an amount ranging from 0.001% to 10% by weight, relative to the total weight of the composition.

32. The composition according to claim 31, wherein the at least one cationic polymer is present in the composition in an amount ranging from 0.005% to 5% by weight, relative to the total weight of the composition.

33. The composition according to claim 1, further comprising at least one nacreous agent and/or opacifier.

34. The composition according to claim 33, wherein the at least one nacreous agent and/or opacifier is chosen from coated and uncoated titanium oxides, sodium palmitate, magnesium palmitate, sodium stearate, sodium hydroxystearate, magnesium stearate, and magnesium hydroxystearate, fatty-chain acyl derivatives, fatty-chain ethers, distearyl ether and 1-(hexadecyloxy)-2-octadecanol, cyclodextrins, and fatty alcohols.

35. The composition according to claim 34, wherein the fatty-chain acyl derivatives are chosen from ethylene glycol monostearate, ethylene glycol distearate, polyethylene glycol monostearate, and polyethylene glycol distearate.

36. The composition according to claim 34, wherein the fatty alcohols are chosen from cetyl alcohol, stearyl alcohol and behenyl alcohol.

37. The composition according to claim 1, further comprising at least one additive chosen from thickeners, fragrances, preserving agents, sunscreens, anionic, nonionic and amphoteric polymers, proteins, protein hydrolysates, ceramides, pseudoceramides, linear and branched C₁₆-C₄₀ fatty acids, vitamins, provitamins, plant oils, mineral oils, synthetic oils and antidandruff agents.

38. The composition according to claim 37, wherein the linear and branched C₁₆-C₄₀fatty acids are chosen from 18-methyleicosanoic acid.

39. The composition according to claim 37, wherein the provitamins are chosen from panthenol.

40. The composition according to claim 1, further comprising at least one foam synergist.

41. The composition according to claim 40, wherein the at least one foam synergist is chosen from C₁₀-C₁₈ 1,2-alkanediols and fatty alkanolamides derived from monoethanolamine or diethanolamine.

42. The composition according to claim 1, the composition is in a form chosen from shampoos; washing compositions for the skin; rinse-out and leave-in conditioners; permanent-waving compositions; hair-relaxing compositions; dyeing compositions; bleaching compositions; rinse-out compositions to be applied before and/or after dyeing, bleaching, permanent-waving and relaxing the hair; and rinse-out compositions to be applied between the two steps of permanent-waving and/or hair-relaxing processes.

43. A method for washing keratin materials, comprising applying to the keratin materials a composition comprising, in a cosmetically acceptable medium, at least one amphoteric surfactant chosen from alkylamphohydroxyalkylsulphonates and salts thereof, and at least 1% by weight, relative to the total weight of the composition, of at least one hydroxy acid.

44. The method according to claim 43, wherein the keratin materials are hair.

45. A composition comprising, in a cosmetically acceptable medium, at least one amphoteric surfactant chosen from alkylamphohydroxyalkylsulphonates and salts thereof, and at least 1% by weight, relative to the total weight of the composition, of at least one hydroxy acid, wherein the at least one amphoteric surfactant and the at least one hydroxy acid are present in a combined amount effective to impart to the hair at least one property chosen from improved disentangling, smoothing, volume, lightness, softness, flexibility and manageability.

46. A method for imparting to the hair at least one property chosen from improved disentangling, smoothing, volume, lightness, softness, flexibility and manageability, comprising applying to the hair an effective amount of a composition comprising, in a cosmetically acceptable medium, at least one amphoteric surfactant chosen from alkylamphohydroxyalkylsulphonates and salts thereof, and at least 1% by weight, relative to the total weight of the composition, of at least one hydroxy acid.

47. A method for treating keratin materials, comprising applying to the materials a cosmetic composition comprising, in a cosmetically acceptable medium, at least one amphoteric surfactant chosen from alkylamphohydroxyalkylsulphonates and salts thereof, and at least 1% by weight, relative to the total weight of the composition, of at least one hydroxy acid, and then optionally rinsing the composition out.

48. The method according to claim 47, wherein the keratin materials are hair.
